(19) United States
(12) Patent Application Publication
(10) Pub. No.: US 2002/0127254 A1
(43) Pub. Date: Sep. 12, 2002

(54) Devices for local and systemic delivery of active substance and methods of manufacturing thereof

(75) Inventors: Spiros Fotinos, Athens (GR); David O 'Halloran, Milltown, NJ (US); Yelena Zolotarsky, Springfield, NJ (US)

Correspondence Address: ALLEN BLOOM, C/O DECHERT, PRINCETON PIKE CORPORATION CENTER, P.O. BOX 5218, PRINCETON, NJ, 08543-5218, US

(73) Assignee: Lavipharm Laboratories Inc.

(21) Appl. No.: 10/036,487

(22) Filed: Jan. 7, 2002

Related U.S. Application Data
(63) Continuation-in-part of application No. 09/340,338, filed on Jun. 25, 1999.
(60) Provisional application No. 60/090,674, filed on Jun. 25, 1998.
(60) Provisional application No. 60/260,587, filed on Jan. 9, 2001.

(51) Int. Cl. A61K 31/5513; A61K 31/19; A61K 9/70

(52) U.S. Cl. 424/401; 424/449; 514/221; 514/557; 514/23

(57) ABSTRACT

Solid gel film compositions for local and systemic delivery of active substances through the skin or mucosal epithelial layer of a subject are provided. More particularly, delivery discs are provided containing a uniform mixture, wherein the uniform mixture contains a filmogenic polymer and a therapeutically effective dose of an active substance, wherein the delivery disc is a single uniform layer device which is non-tacky and which dissolves onto a skin tissue skin tissue with few drops of water or lotion or mucosal epithelial tissue of a subject when applied thereto. Methods for administering an active substance to a subject using a delivery disc of the present invention are provided. Methods for preparing the delivery discs of the present invention are also provided.

This Application is a continuation-in-part of U.S. patent application Ser. No. 09/340,338, filed Jun. 25, 1999 and claims priority from U.S. Provisional Application Serial No. 60/090,674, filed Jun. 25, 1998 and No. 60/260,587, filed Jan. 9, 2001; the disclosures of each of which are incorporated herein by reference as if set forth herein in their entireties.

The present invention relates to solid gel film compositions for local and systemic delivery of active substances through the skin or mucosal epithelial layer of a subject. More particularly, the present invention relates to delivery discs containing a uniform mixture, wherein the uniform mixture contains a filmogenic polymer and a therapeutically effective dose of an active substance for pharmaceutical or cosmetic application, wherein the delivery disc might contain water-soluble dyes, wherein the delivery disc is a single uniform layer device which is non-tacky and which dissolves onto a skin tissue with few drops of water or lotion or mucosal epithelial tissue of a subject when applied thereto. The present invention also relates to a method of administering an active substance to a subject using a delivery disc of the present invention. The present invention further relates to a method or producing the delivery discs of the present invention.

### SUMMARY OF THE INVENTION

In a preferred embodiment of the present invention, a delivery disc containing a uniform mixture is provided, wherein the uniform mixture contains a filmogenic polymer and an effective dose of an active substance, wherein the delivery disc is a single uniform layer device which is non-tacky. In a preferred aspect of the invention, the delivery disc dissolves onto a skin tissue with few drops of water or lotion or mucosal epithelial tissue of a subject when applied thereto.

In a preferred aspect of the present invention, the uniform mixture further contains at least one additive selected from the group of a stabilizer, a solubilizer, a permeation enhancer, a surfactant and a plasticizer.

In another preferred aspect of the present invention, the uniform mixture preferably contains 0.1 to 1 5 wt % active substance. Preferably, the active substance is selected from the group of cosmetic agents and therapeutic agents. Preferably, the cosmetic agents are selected from the group of anti-hyperpigmentation agents, anti-acne agents, keratolytic agents, anti-blotching agents, anti-aging agents, eye contour agents, slimming agents, anti-cellulite agents, soothing/sunburn anti-irritating agents, skin firming and lifting agents, anti-elastase and anti-collagenase substances, free radical scavengers, seboregulators, hydratives and alpha-hydroxy acids. Preferably, the therapeutic agents are selected from the group of cardiovascular agents, hormones, neurotransmitters, antibiotics, antimicrobials, catecholamines and sympathomimetic drugs, adrenergic receptor agonists and antagonists, anesthetics, benzodiazepines, analgesics, antidepressants, hypnotics, sedatives, antipsychotic agents, muscle relaxants and anti-cancer agents.

In another preferred aspect of the present invention, the uniform mixture preferably contains 0.01 to 15 wt % permeation enhancer. Preferably, the permeation enhancer is selected from the group of a glycolipid, a non-esterified fatty acid, an aliphatic alcohol, a fatty acid ester of an aliphatic alcohol, a cyclohexanol, a fatty acid ester of glycerol, a glycol, an aliphatic alcohol ether of a clycol and mixtures thereof.

In another preferred aspect of the present invention, the uniform mixture preferably contains 5 to 100 wt % filmogenic polymer. In one preferred embodiment of the present invention, the uniform mixture preferably contains 5 to 50 wt % filmogenic polymer. In another preferred embodiment, the uniform mixture preferably contains at least 75 wt % filmogenic polymer, in which embodiment the site of application of the delivery disc is preferably prewetted before application of the delivery disc. In another preferred embodiment, the uniform mixture preferably contains 90 to 95 wt % filmogenic polymer. Preferably, the filmogenic polymer is selected from the group of polyvinyl pyrrolidone, chitin, chitosan, xanthan gum, karaya gum, zein, hordein, gliadin and mixtures thereof, most preferably polyvinyl pyrrolidone.

In another preferred aspect of the present invention, the uniform mixture preferably contains 0 to 60 wt % plasticizer. In one preferred embodiment of the present invention, the uniform mixture contains 30 to 60 wt % plasticizer. In another preferred embodiment, the uniform mixture contains less than 30 wt % plasticizer. Preferably, the plasticizer is polyethylene glycol, most preferably polyethylene glycol 400 and polyethylene glycol 4000.

In another preferred aspect of the present invention, the uniform mixture preferably contains 1 to 20 wt % surfactant/emulsifier. Preferably, the surfactant is selected from the group of ethoxylated alcohols, sodium lauryl sulfate and betaine.

In another preferred embodiment of the present invention, a method for transdermally administering an active substance to a subject is provided, which method includes: (a) wetting a skin tissue of the subject at a site of application; and, (b) applying to the site of application a delivery disc of the present invention containing a uniform mixture, wherein the uniform mixture preferably contains a filmogenic polymer and an effective dose of an active substance, wherein the delivery disc is preferably a single uniform layer device which preferably is non-tacky and which preferably dissolves onto the wetted skin tissue or mucosal epithealial tissue of a subject when administered thereto. In this embodiment of the present invention, the delivery discs are preferably administered to a skin tissue with few drops of water or lotion with gentle rubbing or mucosal epithealial tissue.

In another preferred embodiment of the present invention, a method for transmucosally administering an active substance to a subject is provided, which method includes: (a) applying a delivery disc of the present invention to a mucosal epithelial layer of the subject; wherein the delivery disc contains a uniform mixture, wherein the uniform mixture contains a filmogenic polymer and an effective dose of an active substance, wherein the delivery disc is preferably a single uniform layer device which preferably is non-tacky and which preferably dissolves onto the wetted skin tissue or mucosal epithelial tissue of the suject when administered thereto. In this embodiment of the present invention, the delivery discs are preferably administered to a skin tissue with few drops of water or lotion with gentle rubbing or mucosal epithealial tissue.

In another preferred embodiment of the present invention, a method for cleansing a skin tissue of a subject is provided, which method includes: (a) wetting the skin tissue of the subject to be cleansed; and (b) applying a delivery disc to the wetted skin tissue; or vice versa (first applying the disc, and then wetting it) wherein the delivery disc comprises a uniform mixture, wherein the uniform mixture comprises a filmogenic polymer and a surfactant. The delivery disc used in this method of the present invention preferably is non-tacky and preferably dissolves and foams on the wetted skin tissue of the subject when administered thereto. In this embodiment of the present invention, the delivery discs are preferably administered to a skin tissue with water and worked into a lather (foam) with gentle rubbing and then rinsed from the skin.

### DETAILED DESCRIPTION

The term “transdermal” as used herein and in the appended claims means a route of delivery for an active substance across the epithelial layer of a patient, including across the stratum comeum comprising dead differentiated epithelial cells that have produced a dry keratinized layer.

The term “transmucosal” as used herein and in the appended claims means a route of delivery for an active substance across a mucosal epithelial layer, for example, in an oral cavity and a vaginal cavity of a subject.

Transdermal and transmucosal drug delivery systems are described in “Transdermal and Topicl Drug Delivery Systems,” Ed. Ghosh, T. et al. (Buffalo Grove, Ill.: Interpharm Press, Inc., 1997), which describes the higher permeability of mucosal tissue, which reference is incorporated herein by reference as if set forth herein in full.

The present invention provides delivery discs for administering an active substance to a subject and/or for cleansing a skin tissue of a subject. The delivery discs of the present invention contain a uniform mixture containing a filmogenic polymer and an effective dose of an active substance and/or a surfactant. The delivery discs of the present invention dissolves onto a skin tissue with few drops of water or lotion or a mucosal epithelial tissue of a subject when administered thereto.

The delivery discs of the present invention preferably contain a filmogenic polymer and an effective dose of an active substance. The delivery discs of the present invention are preferably a single uniform layer device which is preferably non-tacky and which preferably dissolves onto a skin tissue with few drops of water or lotion or mucosal epithelial tissue of a subject when applied thereto. The delivery discs of the present invention may preferably be biodegradable. The delivery discs of the present invention may be rigid or flexible. Rigid delivery discs of the present invention do not require additional support structures such as backing layers and or release layers.

The uniform mixture used in the delivery discs of the present invention may preferably contain at least one optional additive selected from the group of stabilizers, solubilizers, permeation enhancers, surfactants, plasticizers and water soluble dies.

Active substances suitable for use with the present invention preferably include cosmetic agents and therapeutic agents. Preferably, the delivery discs of the present invention may contain 0.1 to 1 5 wt % active substances. The active substances may be incorporated into a carrier system. Carrier systems suitable for use with the present invention include, but are by no means limited to, liposomes and cyclodextrin complexes.

Cosmetic agents suitable for use with the present invention include, but are by no means limited to, anti-hyperpigmentation agents, anti-acne, keratolytic, anti-blotching agents, anti-aging agents, eye contour agents, slimming agents, anti-cellulite agents, soothing/sunburn anti-irritating agents, skin firming and lifting agents, anti-elastase and anti-collagenase substances, free radical scavengers, seboregulators, hydratives and alpha-hydroxy acids.

Therapeutic agents suitable for use with the present invention include, but are by no means limited to, cardiovascular agents, hormones, neurotransmitters, antibiotics, antimicrobials, catecholamines and sympathomimetic drugs, adrenergic receptor agaonists and antagonists, anesthetics, benzodiasepines, analgesics, antidepressants, hypnotics, sedatives, antipsychotic agents, muscle relaxants and anti-cancer agents.

Filmogenic polymers suitable for use with the present invention include, but are by no means limited to, any synthetic polymers with filmogenic properties, any natural polymers with filmogenic properties and mixtures thereof. Preferably, the filmogenic polymer may be selected from the group of polyvinyl pyrrolidone, chitin, chitosan, xanthan gum, karaya gum, zein, hordein, gliadin and mixtures thereof; preferably polyvinyl pyrrolidone. Preferably, the delivery discs of the present invention may preferably contain 5 to 100 wt % filmogenic polymer, preferably 5 to 50 wt % filmogenic polymer, alternatively preferably at least 75 wt % filmogenic polymer.

Plasticizers suitable for use with the present invention include, but are by no means limited to, any plasticizers conventionally used in transdermal and transmucosal delivery devices. Preferred plasticizers may include polyethylene glycol, more preferably polyethylene glycol—4000. Preferably, the delivery discs of the present invention may contain 0 to 60 wt % plasticizer. In one preferred embodiment of the present invention, the uniform mixture may contain 30 to 60 wt % plasticizer. In another preferred embodiment, the delivery discs of the present invention may contain less than 30 wt % plasticizer.

Delivery discs using a uniform mixture containing less than 50 wt % filmogenic polymer preferably contain 30 to 60 wt % plasticizer. Delivery discs using a uniform mixture containing at least 75 wt % filmogenic polymer preferably contain less than 30 wt % plasticizer. Prewetting of a skin tissue at a non-mucosal epithelial tissue site of administration of a delivery discs of the present invention may not be necessary when the uniform mixture contains 30 to 60 wt % plasticizer. Prewetting is generally required, however, when the uniform mixture contains less than 30 wt % plasticizer.

Permeation enhancers suitable for use with the present invention include, but are by no means limited to, glycolipids, non-esterified fatty acids, aliphatic alcohols, fatty acid esters of aliphatic alcohols, cyclohexanol, fatty acid esters of glycerols, glycols, aliphatic alcohol ethers of a glycol and mixtures thereof. Preferably, the delivery discs of the present invention may contain 0.01 to 15 wt % permeation enhancers.

Surfactants suitable for use with the present invention include, but are by no means limited to, any anionic, cationic or nonionic surfactants or emulsifiers conventionally used in cosmetics. Preferred surfactants may include ethoxylated alcohols, sodium lauryl sulfate and betaine. Preferably, the delivery discs of the present invention may contain 1 to 20 wt % surfactants.

Water soluble dies suitable for use with the present invention include, but are by no means limited to, any water soluble dies conventionally used in cosmetics. Preferred water soluble dies may include FD&C and D&C Dyes. Preferably, the delivery discs of the present invention may contain 0.001 to 2.00 wt % water soluble dies. The use of water soluble dies in the delivery discs of the present invention provide the opportunity to add fun to the application of the given discs. That is, the delivery discs may be colored using these water soluble dies such that the color dissappears as the delivery disc dissolves into the skin. This aspect of the invention provides vast opportunity to advance marketing concepts in a fun way.

The present invention further provides a method for transdermally administering an active substance to a subject, which method includes: (a) wetting a skin tissue of the subject at a site of application; and, (b) applying to the site of application a delivery disc of the present invention containing a uniform mixture, wherein the uniform mixture preferably contains a filmogenic polymer and an effective dose of an active substance, wherein the delivery disc is preferably a single uniform layer device which preferably is non-tacky and which preferably dissolves onto a skin tissue with few drops of water or mucosal epithealial tissue of a subject when administered thereto. In this embodiment of the present invention, the delivery discs are preferably administered to a skin tissue with few drops of water/lotion with gentle rubbing or mucosal epithealial tissue.

The present invention further provides a method for transmucosally administering an active substance to a subject, which method includes: (a) applying a delivery disc of the present invention to a mucosal epithelial layer of the subject; wherein the delivery disc contains a uniform mixture, wherein the uniform mixture contains a filmogenic polymer and an effective dose of an active substance, wherein the delivery disc is preferably a single uniform layer device which preferably is non-tacky and which preferably dissolves onto skin tissue with few drops of water or mucosal epithelial tissue of the subject when administered thereto. In this embodiment of the present invention, the delivery discs are preferably administered to a skin tissue with few drops of water or lotion with gentle rubbing or mucosal epithealial tissue.

The present invention further provides a method for cleansing a skin tissue of a subject, which method includes: (a) wetting the skin tissue of the subject to be cleansed; and (b) applying a delivery disc to the wetted skin tissue or vice versa (first applying the disc, and then wetting it); wherein the delivery disc comprises a uniform mixture, wherein the uniform mixture comprises a filmogenic polymer and a surfactant. The delivery disc used in this method of the present invention preferably is non-tacky and preferably dissolves and foams on wetted skin tissue of the subject when administered thereto. In this embodiment of the present invention, the delivery discs are preferably administered to a skin tissue with water and then worked into a lather (foam) and then rinsed from the skin.

### EXAMPLES

The preferred embodiments of the present invention will now be further described through the following examples set forth here in below which are intended to be illustrative of the preferred embodiments of the present invention and are not intended to limit the scope of the invention as set forth in the appended claims.

### Examples 1-5

Five example formulations for preferred delivery discs of the present invention are set forth in Table 1.

TABLE 1

| | Ex. 1 | Ex. 2 | | | |
|---|---|---|---|---|---|
| | (wt % | (wt % | Ex. 3 | Ex. 4 | Ex. 5 |
| Ingredient | dry) | dry) | (wt % dry) | (wt % dry) | (wt % dry) |
| Polyvinyl | 86.0 | 88.3 | 86.0 | 86.0 | 80.0 |
| pyrrolidone | | | | | |
| Polyethylene | 5.5 | 0.9 | 5.5 | 5.5 | 5.5 |
| glycol-4000 | | | | | |
| Butylene glycol | | 3.0 | | | |
| Glycerin | | 3.0 | | | |
| Spheron L-1500 | 3.0 | | 3.0 | 3.0 | 3.0 |
| Silica | | | | | |
| Laponite XL | | 1.0 | | | |
| Sodium | | | | | |
| Magnesium | | | | | |
| Silicate | | | | | |
| Menthol | | | | | 10.0 |
| Salicylic acid | | | 4.0 | | |
| Ascorbic acid | | | | 4.0 | |
| Lactic acid | 4.0 | | | | |
| Avalure UR- | | 2.0 | | | |
| 410 | | | | | |
| Polyurethane | | | | | |
| Nylon 66 | | 4.8 | | | |
| Water | QS | QS | QS | QS | QS |

### Example 1

The uniform mixture formulation identified in Table 1 as Example 1 was prepared as follows:

(a) dissolving the polyvinyl pyrrolidone in a volume of alcohol;

(b) slowly adding the polyethylene glycol into the product of (a);

(c) dissolving the lactic acid into a volume of water;

(d) adding the butylene glycol to the product of (c);

(e) adding the product (d) to the product of (b);

(f) removing air from the product of (e);

(g) adding the silica to the product of (f) mix until uniform;

(h) casting the product of (g) onto a release liner at 1 5mil thickness; and,

(i) drying the product of (h) in an oven until uniform and non-tacky (about 10 minutes).

### Example 2

The uniform mixture formulation identified in Table 1 as Example 2 was prepared as follows:

(a) adding laponite XL into a volume of water under vigorous agitation until a clear gel with no entrained particles is formed;

(b) adding polyvinyl pyrrolidone into a volume of alcohol with agitation;

(c) adding the polyethylene glycol to the product of (b) and mixing until uniform

(d) mixing the products of (a) and (c)

(e) blending the avalure with the glycerin and a volume of water until uniform;

(f) adding the product of (e) to the product of (d);

(g) removing air from the product of (f);

(h) adding the nylon 66 to the product of (g);

(i) casting the product of (h) onto a release liner at 1 5-mil thickness; and,

(j) drying the product of (i) in an oven until the film is uniform and not-tacky (about 10 minutes).

### Example 3

The uniform mixture formulation identified in Table 1 as Example 3 was prepared as follows:

(a) adding the polyvinyl pyrrolidone to a volume of alcohol;

(b) adding the polyethylene glycol to the product of (a);

(c) blending the salicylic acid, the butylene glycol and a volume of water and adding the blend to the product of (b);

(d) removing air from the product of (c);

(e) adding silica to the product of (d);

(f) casting the product of (e) onto a release liner at 1 5-mil thickness; and

(g) drying the product of (f) in an oven until the film is uniform and not tacky (about 10 minutes).

### Example 4

The uniform mixture formulation identified in Table 1 as Example 4 was prepared as follows:

(a) adding polyvinyl pyrrolidone to a volume of alcohol;

(b) adding the polyethylene glycol to the product of (a);

(c) dissolving ascorbic acid into a volume of water;

(d) adding butylene glycol to the product of (c);

(e) mixing the products of (b) and (d);

(f) removing air from the product of (e);

(g) adding silica to the product of (f);

(h) casting the product of (g) onto a release liner at 1 5-mil thickness; and,

(i) drying the product of (h) in an over until the film is uniform and non-tacky (about 10 minutes).

### Example 5

The uniform mixture formulation identified in Table 1 as Example 5 was prepared as follows:

(a) adding polyvinyl pyrrolidone to a volume of alcohol;

(b) adding polyethylene glycol to the product of (a);

(c) dissolving the menthol in the butylene glycol and a volume of water;

(d) mixing the products of (b) and (c);

(e) removing air from the product of (d);

(f) adding silica to the product of (e);

(g) casing the product of (f) on a release liner at 15-mil thickness; and,

(h) drying the product of (g) in an over until the film is uniform and non-tacky (about 10 minutes).

### Examples 6-8 (Cleansing Solid Gels)

The example formulations for preferred cleansing gel delivery discs of the present invention are set forth in Table 2.

TABLE 2

| | Ex. 6 | Ex. 7 | Ex. 8 |
|---|---|---|---|
| Ingredient | (wt % dry) | (wt % dry) | (wt % dry) |
| Polyvinyl pyrrolidone | 78.0 | 81.3 | 80.0 |
| (K-90) | | | |
| Polyethylene glycol 4000 | 5.5 | 0.9 | 5.5 |
| Butylene glycol | 1.5 | | 1.5 |
| Glycerin | | 3.0 | |
| Spheron L-1500 Silica | 3.0 | | 3.0 |
| Laponite XL (Sodium | | 1.0 | |
| Magnesium Silicate) | | | |
| Salicylic acid | | | 4.0 |
| Lactic acid | 2.0 | | |
| Avalure UR-410 | | 2.0 | |
| polyurethane | | | |
| Nylon 66 | | 4.8 | |
| Surfadone LP (Lauryl | | | 1.0 |
| Pyrrolidone | | | |
| Betaine | | 2.0 | |
| Alkyl (C12, C10, C16) | 3.0 | | |
| Glucoside | | | |
| Magnesium Laureth | | 5.0 | |
| Sulfate | | | |
| Sodium Lauryl sulfate | 7.0 | | 5.0 |
| Water | QS | QS | QS |

### Example 6

The uniform mixture formulation identified in Table 2 as Example 6 was prepared as follows:

(a) adding the polyvinyl pyrrolidone to ethyl alcohol blending well;

(b) adding the polyethylene glycol to the product of (a);

(c) adding the lactic acid and the butylene glycol to the product of (b);

(d) adding the alkyl glucoside and sodium lauryl sulfate to the product of (c);

(e) removing air from the product of (d);

(f) adding silica to the product of (e);

(g) casting the product of (f) onto a release liner at 1 5-mil thickness; and, (h) drying the product of (g) in an oven to produce a uniform non-tacky film.

### Example 7

The uniform mixture formulation identified in Table 2 as Example 7 was prepared as follows:

(a) adding the polyvinyl pyrrolidone to ethyl alcohol, then adding to this mixture the Laponite XL (which is dispersed into a volume of water under vigorous agitation);

(b) adding the magnesium laureth sulfate to the product of (a);

(c) adding the polyethylene glycol to the product of (b);

(d) blending avalure and glycerin until uniform;

(e) mixing the products of (c) and (d);

(f) adding betaine to the product of (e);

(g) removing air from the product of (f);

(h) adding nylon 66 to the product of (g);

(i) casting the product of (h) onto a release liner at 1 5-mil thickness; and,

(j) drying the product of (i) until the film is uniform and non-tacky (about 10 minutes).

### Example 8

The uniform mixture formulation identified in Table 2 as Example 7 was prepared as follows:

(a) mixing the polyvinyl pyrrolidone into ethanol;

(b) adding the polyethylene glycol and sodium laureth sulfate to the product of (a);

(c) dissolving the salicylic acid in the butylene glycol;

(d) mixing the products of (b) and (c);

(e) removing air from the product of (d);

(f) adding silica to the product of (e);

(g) adding surfadone to the product of (f);

(h) casting the product of (g) onto a release liner at 15-mil thickness; and,

(i) drying the product of (h) in an oven until the film is uniform and non-tacky (about 10 minutes).

### Examples 9-14 (Devices for Local/Systemic Delivery of Active Substances)

The example formulations for preferred cleansing gel delivery discs of the present invention are set forth in Table 3.

TABLE 3

| | Ex. 9 | Ex. 10 | Ex. 11 | Ex. 12 | Ex. 13 | Ex. 14 |
|---|---|---|---|---|---|---|
| | (wt % | (wt % | (wt % | (wt % | (wt % | (wt % |
| Ingredient | dry) | dry) | dry) | dry) | dry) | dry) |
| Hormone | 6.20 | 6.20 | | | | 6.20 |
| Linoleic acid¹ | 6.00 | | | | | |
| Lipocol-12² | | 8.00 | | | | |
| Isopropyl myristate | | | 3.70 | 3.70 | | |
| Cosmetic agent | | | 2.50 | 2.50 | 2.50 | |
| Eutanol ZG16S³ | | | 2.50 | | 2.50 | |
| L-Menthol⁴ | | | 5.00 | 5.00 | | |
| Montane 80 VGA⁵ | | | | | | 1.5 |
| Gliadin mixture⁶ | | | | | | 93.30 |
| Polyvinly pyrrolidone⁷ | 42.40 | 41.50 | 41.70 | 42.90 | 91.00 | |
| Polyethylene glycol - 400⁸ | 45.40 | 44.30 | 44.60 | 45.90 | 4.00 | |

¹Linoleic acid is a fatty acid commercially available from Croda Chemicals Ltd.
²Lipocol-12 is 12 mole polyethylene glycol ether of lauryl alcohol commercially available from LIPO Chemicals Inc.
³Eutanol G 16S is hexyldecyl stearate commercially available from Henkel KgaA.
⁴L-Menthol is commercially available from Sigma Chemical Co.
⁵Montane 80 VGA is polysorbate monooleate commercially available from Seppic.
⁶Gliadin mixture is commercially available from Inocosm Lab.
⁷Polyvinyl pyrrolidone is commercially available from BASF Aktiengesellschaft under the trademark “Kollidon 90 F”.
⁸Polyethylene glycol 400 is commercially available from ICI Surfactants.

The present invention having been disclosed in connection with the foregoing embodiments, additional embodiments will now be apparent to persons skilled in the art. The present invention is not intended to be limited to the embodiments specifically mentioned, and accordingly reference should be made to the appended claims rather than the foregoing discussion, to assess the spirit and scope of the present invention in which exclusive rights are claimed.

We claim:

1. A delivery disc comprising a uniform mixture, wherein the uniform mixture comprises a filmogenic polymer and an effective dose of an active substance, wherein the delivery disc is a single uniform layer device which is non-tacky and which dissolves onto a wetted skin tissue or mucosal epithelial tissue of a subject when applied thereto.

2. The delivery disc of claim 1, wherein the uniform mixture further comprises at least one additive selected from the group consisting of a stabilizer, a solubilizer, a permeation enhancer, a surfactant and a plasticizer.

3. The delivery disc of claim 1, wherein the active substance is selected from the group consisting of a cosmetic agent and a therapeutic agent.

4. The delivery disc of claim 3, wherein the cosmetic agent is selected from the group consisting of: anti-hyperpigmentation agents, anti-blotching agents, anti-aging agents, eye contour agents, slimming agents, anti-cellulite agents, soothing/sunburn anti-irritating agents, skin firming and lifting agents, anti-elastase and anti-collagenase substances, free radical scavengers, seboregulators, hydratives and alpha-hydroxy acids.

5. The delivery disc of claim 3, wherein the cosmetic agent is selected from the group consisting of anti-acne agents.

6. The delivery disc of claim 3, wherein the therapeutic agent is selected from the group consisting of: cardiovascular agents, hormones, neurotransmitters, antibiotics, antimicrobials, catecholamines and sympathomimetic drugs, adrenergic receptor agonists and antagonists, anesthetics, benzodiazepines, analgesics, antidepressants, hypnotics, sedatives, antipsychotic agents, muscle relaxants and anti-cancer agents.

7. The delivery disc of claim 2, wherein the permeation enhancer is selected from the group consisting of a glycolipid, a non-esterified fatty acid, an aliphatic alcohol, a fatty acid ester of an aliphatic alcohol, a cyclohexanol, a fatty acid ester of glycerol, a glycol, an aliphatic alcohol ether of a glycol and mixtures thereof.

8. The delivery disc of claim 1, wherein the filmogenic polymer is selected from the group consisting of polyvinyl pyrrolidone, chitin, chitosan, xanthan gum, karaya gum, zein, hordein, gliadin and mixtures thereof.

9. The delivery disc of claim 2, wherein the plasticizer is polyethylene glycol 4000.

10. The delivery disc of claim 2, wherein the surfactant is selected from the group consisting of ethoxylated alcohols, sodium lauryl sulfate and betaine.

11. The delivery disc of claim 10, wherein the uniform mixture comprises 1 to 20 wt % surfactant.

12. The delivery disc of claim 2, wherein the uniform mixture comprises less than 30 wt % plasticizer.

13. The delivery disc of claim 1, wherein the uniform mixture comprises at least 5 wt % filmogenic material.

14. The delivery disc of claim 13, wherein the uniform mixture comprises less than 50 wt % filmogenic material.

15. The delivery disc of claim 13, wherein the uniform mixture comprises at least 75% filmogenic material.

16. The delivery disc of claim 2, wherein the uniform mixture comprises less to 60 wt % plasticizer.

17. The delivery disc of claim 2, wherein the uniform mixture comprises less than 30 wt % plasticizer.

18. The delivery disc of claim I, wherein the uniform mixture comprises 0.1 to 15 wt % active substance.

19. The delivery disc of claim 1, wherein the uniform mixture comprises 0.01 to 15 wt % permeation enhancer.

20. A method for transdermally administering an active substance to a subject comprising:
(a) wetting a skin tissue of the subject at a site of application; and,
(b) applying to the site of application a delivery disc;
wherein the delivery disc comprises a uniform mixture, wherein the uniform mixture comprises a filmogenic polymer and an effective dose of an active substance, wherein the delivery disc is a single uniform layer device which is non-tacky and which dissolves onto a wetted skin tissue or mucosal epithelial tissue of a subject when applied thereto.

21. A method for transmucosally administering an active substance to a subject comprising:
(a) applying a delivery disc to a mucosal epithelial layer of the subject; wherein the delivery disc comprises a uniform mixture, wherein the uniform mixture comprises a filmogenic polymer and an effective dose of an active substance, wherein the delivery disc is a single uniform layer device which is non-tacky and which dissolves onto a wetted skin tissue or mucosal epithelial tissue of a subject when applied thereto.

22. A method for cleansing a skin tissue of a subject comprising:
(a) wetting the skin tissue of the subject to be cleansed; and,
(b) applying a delivery disc to the wet skin tissue;
wherein the delivery disc comprises a uniform mixture, wherein the uniform mixture comprises a filmogenic polymer and a surfactant.

23. The method of claim 22, further comprising:
(c) creating a foam or lather with the delivery disc on the wet skin tissue and rinsing from the skin.
